(19) United States
(12) Patent Application Publication
(10) Pub. No.: US 2005/0277994 A1
(43) Pub. Date: Dec. 15, 2005

(54) Apparatus and method for estimating battery condition in implantable cardiac devices

(76) Inventors: Paul McNamee, St. Paul, MN (US); John A. Dyjach, Circle Pines, MN (US); Kristofer J. James, Eagan, MN (US)

Correspondence Address: SCHWEGMAN, LUNDBERG, WOESSNER & KLUTH, 1600 TCF TOWER, 121 SOUTH EIGHT STREET, MINNEAPOLIS, MN, 55402, US

(21) Appl. No.: 10/996,339

(22) Filed: Nov. 23, 2004

Related U.S. Application Data
(63) Continuation-in-part of application No. 10/864,759, filed on Jun. 9, 2004, now abandoned.

(51) Int. Cl. A61N 1/37

(52) U.S. Cl. 607/29

(57) ABSTRACT

An apparatus and method for determining the condition of a battery in an implantable cardiac rhythm management device is described. A battery's status is determined from a record of the device's operational history. The operational history may include the total number of events or event durations recorded during a specified time period. The battery charge consumption is then estimated by means of charge coefficients associated with each type of event.

### RELATED APPLICATION

This application is a continuation-in-part and claims priority of invention under 35 U.S.C. §120 from U.S. application Ser. No. 10/864,759, filed Jun. 9, 2004, which is incorporated herein by reference.

### FIELD OF THE INVENTION

This invention pertains to systems and methods for operating battery-powered implantable medical devices.

### BACKGROUND

Cardiac rhythm management devices (CRMDs) are implantable devices that provide electrical stimulation to selected chambers of the heart in order to treat disorders of cardiac rhythm. A pacemaker, for example, is a cardiac rhythm management device that paces the heart with timed pacing pulses. The most common condition for which pacemakers are used is in the treatment of bradycardia, where the ventricular rate is too slow. Atrio-ventricular conduction defects (i.e., AV block) that are permanent or intermittent and sick sinus syndrome represent the most common causes of bradycardia for which permanent pacing may be indicated. If functioning properly, the pacemaker makes up for the heart's inability to pace itself at an appropriate rhythm in order to meet metabolic demand by enforcing a minimum heart rate and/or artificially restoring AV conduction. Other cardiac rhythm management devices are designed to detect atrial and/or ventricular tachyarrhythmias and deliver electrical stimulation in order to terminate the tachyarrhythmia in the form of a cardioversion/defibrillation shock or anti-tachycardia pacing. Certain combination devices may incorporate all of the above functionalities.

CRMDs are powered by a battery contained within the housing of the device that has a limited life span. When the battery fails, the device must be replaced which necessitates a reimplantation procedure. The useful life of the battery may vary in each individual case and depends upon the specific battery and the power requirements of the device. For example, a device which must deliver paces and/or defibrillation shocks on a frequent basis will shorten the useful life of the battery. As the battery depletes, it is desirable to provide a means of determining that the battery is near the end of its life so that replacement of the battery can be scheduled rather than done on an emergency basis.

### BRIEF DESCRIPTION OF THE DRAWINGS

FIG. 1 is a system diagram of an implantable cardiac rhythm management device.

FIG. 2 is a diagram of a capacitor charging circuit.

FIG. 3 illustrates an exemplary embodiment of the method for monitoring and battery status.

### DETAILED DESCRIPTION

The remaining useful life of any particular battery in an implantable device is related to the battery's depth of discharge. One way to determine the depth of discharge is to directly measure the total charge consumed by the device from the battery over a period of time. Such battery charge consumption may be measured by a hardware-based coulomb counter which integrates charge over a particular period of time. This approach, however, has a number of disadvantages, however, including the need for specialized dedicated hardware which precludes adoption on platforms not already provisioned for the measurement. Also, certain devices exhibit a very wide dynamic current range that must be overcome in the design. Lastly, approaches which measure actual charge consumption do not account for battery self-discharge.

Another approach for estimating depth of discharge is by measuring particular battery operating parameters such as the battery voltage and capacitor charge time. Use of such measurements to provide an accurate representation of depth of battery discharge is problematic over certain ranges and for certain battery chemistries. Such measurements may provide limited sensitivity over significant ranges of the battery life thus limiting the precision with which battery status can be reported to the user. For example, the Li—MnO₂ battery chemistry characteristics are such that these types of measures provide a relatively insensitive indication of depth of battery discharge over a significant portion of early battery life.

The present disclosure relates to a method for estimating battery charge consumption in an implantable cardiac device based on a characterization of the device's operational activities over a period of time. This approach can thus be implemented in a manner which is independent of battery characteristics and may be used for a variety of different battery chemistries. Estimating charge consumption in this manner also requires no specialized hardware and accounts for battery self-discharge. Set forth below are a description of an exemplary system which may be programmed to estimate the charge consumption of an implantable device based upon operational history and a description of specific embodiments.

1. Exemplary Device Description

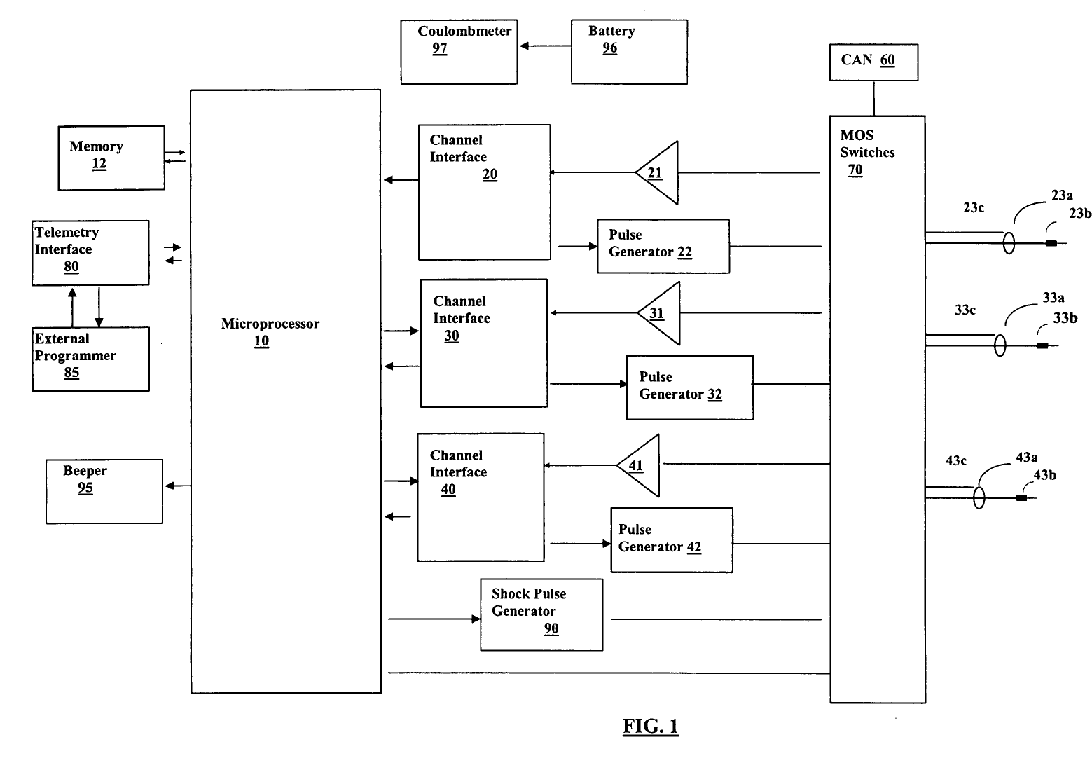

FIG. 1 is a system diagram of an exemplary microprocessor-based cardiac rhythm management device with the capability of delivering cardioversion/defibrillation shocks as well as delivering anti-tachycardia pacing therapy to either the ventricles or the atria. The device may also be configured to deliver conventional (e.g., bradycardia) pacing as well. Such devices are usually implanted subcutaneously on the patient's chest and connected to electrodes by leads threaded through the vessels of the upper venous system into the heart. An electrode can be incorporated into a sensing channel that generates an electrogram signal representing cardiac electrical activity at the electrode site and/or incorporated into a pacing or shocking channel for delivering pacing or shock pulses to the site.

A battery 96 supplies power to the electronic components and is connected to a coulombmeter 97 for direct measurement of charge consumption. The controller of the device is made up of a microprocessor or CPU 10 communicating with a memory 12 via a bidirectional data bus, where the memory 12 typically comprises a ROM (read-only memory) for program storage and a RAM (random-access memory) for data storage. The controller could be implemented by other types of logic circuitry (e.g., discrete components or programmable logic arrays) using a state machine type of design, but a microprocessor-based system is preferable. As used herein, the programming of a controller should be taken to refer to either discrete logic circuitry configured to perform particular functions or to executable code stored in memory or other storage medium. The controller is capable of operating the device so as to deliver a number of different therapies in response to detected cardiac activity. A telemetry system 80 is also provided for enabling the controller to communicate with an external programmer 85 or other device via a wireless telemetry link. The telemetry system in this particular device includes both an inductive telemetry unit for near-field wireless communications and a radio-frequency (RF) transceiver for far-field communications. An audible beeper 95 is also interfaced to the controller for providing a patient with an audible alarm when particular events are detected by the device.

The device shown in FIG. 1 has three sensing/pacing channels, where a pacing channel is made up of a pulse generator connected to an electrode while a sensing channel is made up of the sense amplifier connected to an electrode. A MOS switch matrix 70 controlled by the microprocessor is used to switch the electrodes from the input of a sense amplifier to the output of a pulse generator. The switch matrix 70 also allows the sensing and pacing channels to be configured by the controller with different combinations of the available electrodes. A shock pulse generator 90 is also interfaced to the controller for delivering defibrillation shocks between an electrode and the housing or can 60 as selected by the switch matrix. In an example configuration, a sensing/pacing channel may include ring electrode 43a (33a or 23a) and tip electrode 43b (33b or 23b) of bipolar lead 43c (33c or 23c), sense amplifier 41 (31 or 21), pulse generator 42 (32 or 22), and a channel interface 40 (30 or 20). The channel interfaces communicate bi-directionally with a port of microprocessor 10 and may include analog-to-digital converters for digitizing sensing signal inputs from the sensing amplifiers, registers that can be written to for adjusting the gain and threshold values of the sensing amplifiers, and registers for controlling the output of pacing pulses and/or changing the pacing pulse amplitude. In the illustrated embodiment, the device is equipped with bipolar leads that include two electrodes which are used for outputting a pacing pulse and/or sensing intrinsic activity. Other embodiments may employ unipolar leads with single electrodes for sensing and pacing which are referenced to the device housing or can 60 (or another electrode) by the switch matrix 70. The channels may be configured as either atrial or ventricular channels so as to enable either biatrral or biventricular pacing. For example, a configuration for biventricular sensing/pacing could have one lead of a channel disposed in the right ventricle for right ventricular sensing/pacing and another lead of a channel disposed in the coronary sinus for left ventricular sensing/pacing.

The controller 10 controls the overall operation of the device in accordance with programmed instructions stored in memory, including controlling the delivery of paces via the pacing channels, interpreting sense signals received from the sensing channels, and implementing timers for defining escape intervals and sensory refractory periods. The sensing circuitry of the pacemaker detects a chamber sense when an electrogram signal (i.e., a voltage sensed by an electrode representing cardiac electrical activity) generated by a particular channel exceeds a specified intrinsic detection threshold. A chamber sense may be either an atrial sense or a ventricular sense depending on whether it occurs in the atrial or ventricular sensing channel. By measuring the intervals between chamber senses, the device is able to determine an atrial or ventricular rate, and pacing algorithms used in particular pacing modes employ such senses to trigger or inhibit pacing. Both bradycardia and anti-tachycardia pacing modes may be implemented in code executed by the controller.

2. Battery Voltage and Capacitor Charge Time Measurement

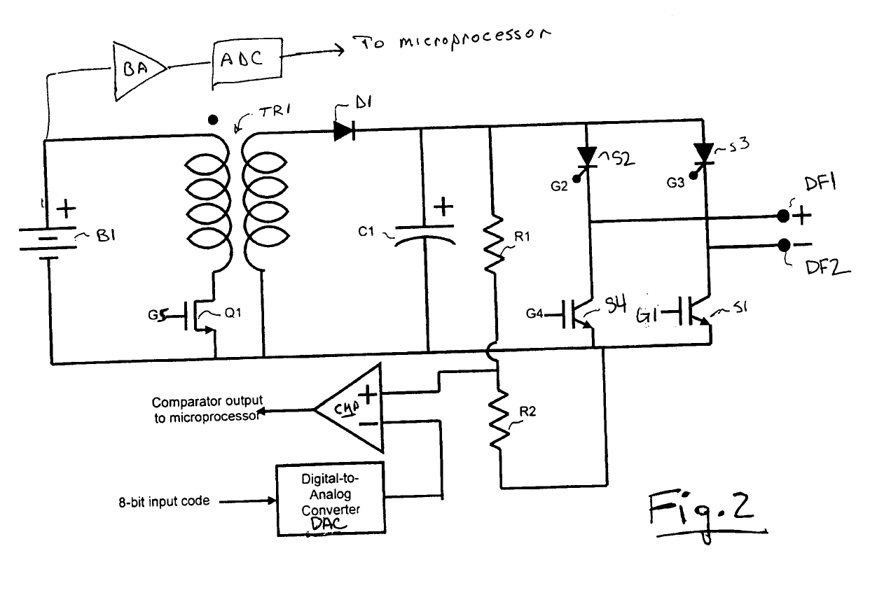

CRMDs typically use an electrolytic output capacitor that is charged from a battery with an inductive boost converter to deliver a shock pulse. When ventricular fibrillation is detected, the CRMD charges up the capacitor to a predetermined value for delivering a shock pulse of sufficient magnitude to convert the fibrillation (i.e., the defibrillation threshold). The capacitor is then connected to the shock electrodes disposed in the heart to deliver the shock pulse. FIG. 2 shows the components of the shock pulse generator and capacitor charging circuitry in more detail. The shock electrodes are connected to defibrillation terminals DF1 and DF2 which are switchably connected to an output capacitor C1 by switches S1 through S4 in a so-called H-configuration. When a shock pulse is delivered, the defibrillation terminals are connected by the aforementioned switches to the capacitor C1 to thereby impress the capacitor voltage across the shock electrodes. Switches S1 through S4 are solid-state device having gate voltages G1 through G4, respectively, that are controlled by the microprocessor 10. By controlling the gate voltages of the switches, the microprocessor can control the polarity of the shock pulse delivered to the electrodes as well as deliver monophasic or biphasic shock waveforms.

The output capacitor C1 is charged from battery B1 to a specified voltage by a charging circuit before each defibrillation shock and during a capacitor reforming procedure. The charging circuit in this embodiment is a boost converter which includes a transformer TR1 and a transistor switch Q1. Transistor Q1 is an FET having its gate voltage G5 connected to the output of an oscillator and includes circuitry for monitoring the drain current to avoid saturating the transformer core. The oscillator (not shown) outputs pulses to switch current on and off in the primary coil of the transformer TR1 and is controlled by the microprocessor 10. The width and/or frequency of the oscillator pulse output may also be controlled in accordance with the primary coil current sensed by transistor Q1. The coils of the transformer TR1 are coupled inductors that receive current from battery B1 during short intervals as dictated by the state of transistor Q1. When transistor Q1 is switched off, the energy stored in the inductance of the transformer is transferred to the capacitor C1 through a diode D1. The capacitor voltage is monitored by circuitry that includes a voltage divider, made up of resistors R1 and R2, and a comparator CMP. The voltage divider feeds the capacitor voltage to the comparator CMP where it is compared with a reference voltage specified by the microprocessor through digital-to-analog converter DAC. The comparator output is then input to the microprocessor which controls the operation of the boost converter to charge the capacitor voltage to a specified level. The microprocessor determines the capacitor charge time by measuring the time from the beginning of the charging cycle until the output of the comparator indicates that the capacitor has been charged to the reference voltage. A buffer amplifier BA and analog-to-digital converter ADC are also provided for measuring the battery voltage.

As a battery in an implantable device progressively depletes, two parameters are affected: the open circuit voltage of the battery decreases and the battery's internal resistance increases. The battery's internal resistance is related to the time required to charge the defibrillation capacitor. The battery voltage and capacitor charge time may be measured as described above, and both of these are useful in estimating the total charge consumption of the battery. Another hardware-based method of estimating battery charge consumption is the use of a coulombmeter such as shown in FIG. 1.

3. Estimating Battery Charge Consumption From Operational History

A system for estimating battery charge consumption and determining battery status may be made up of an implantable device such as illustrated in FIG. 1, which is programmed to record an operational history of the device and compute the battery charge consumption therefrom. Alternatively, the system may be made up of an implantable device in communication with an external programmer which is programmed to estimate battery charge consumption based upon an operational history downloaded to it from the implantable device via a telemetry link. The operational history includes the number of occurrences or durations of selected events, where each type of event may be associated with a particular quantity of charge consumption by a charge coefficient. The charge coefficients for each event type may be determined empirically for a particular device and battery by direct measurement of charge consumption during a device testing procedure prior to implantation. In order to compute the total charge consumption, each such recorded event number or duration is multiplied by the charge coefficient corresponding to the type of event, and the resulting products are summed. The charge coefficients can be either constant or may vary with event number or event duration, the latter case being equivalent to a non-linear mapping of the event number or duration to a particular amount of charge consumption and former being equivalent to a linear mapping. Such linear or non-linear mappings may also be implemented by a look-up table. Also, as described below, the charge coefficients may vary as particular operating parameters of the implantable device are changed.

The system may present to the user an indication of the current battery status as determined from the device operational history either in addition to or in place of hardware-based techniques for determining battery status such as those based upon battery voltage, capacitor charge time, and coulombmeter measurements. In one example embodiment, the system selects for presentation to the user the worst case of battery status as determined by all of the available modalities.

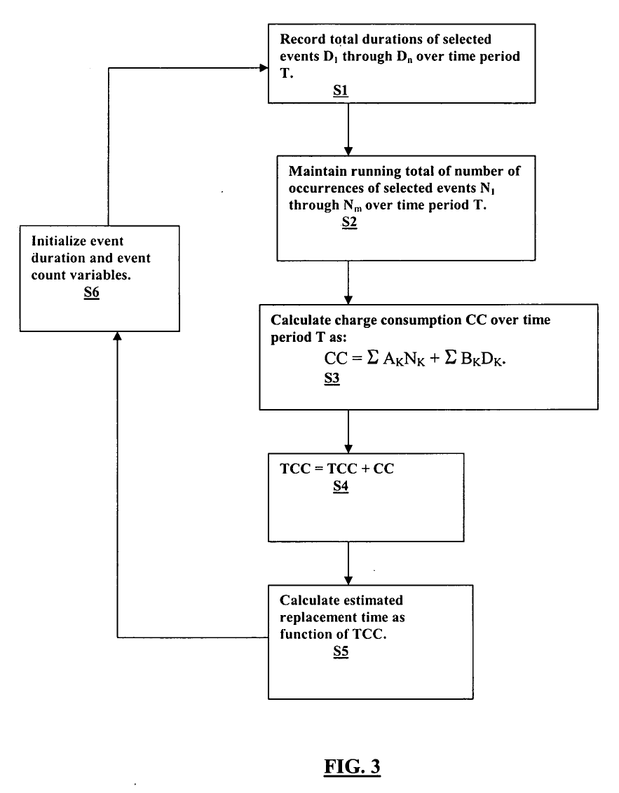

FIG. 3 illustrates a particular example of the method for estimating battery charge consumption as would be implemented by appropriate programming of the device controller and/or external programmer. At step S1, the total time durations of selected events detected by the device, designated as D₁ through Dₙ, are measured over a certain time period T. The time period T may be any selected period such as a day, month, or week. Examples of such event durations could include the total duration of the pacing pulses delivered during the time period T (i.e., the total number of paces delivered during the time period T multiplied by the pulse width), the cumulative charge time used to charge the output capacitor when delivering defibrillation shocks, the measured time during which an inductive telemetry unit was active, the measured time during which a minute ventilation or temperature sensor was active, the measured time during which the device was in a triggered data storage mode, the measured time during which an RF transceiver was active, the measured time during which a beeper was active, the elapsed time since the device was powered up, the time during which the CPU was in an active or awakened state, total duration of tachycardia episodes during which ATP therapy was delivered, and/or the duration of time in which the device was in a low-power storage mode prior to implantation. At step S2, the device maintains a running total of the number of occurrences of selected events, designated as N₁ through Nₘ, over the time period T. Examples of such event counts could include the number of sensed events, the number of paces delivered in a bradycardia pacing mode, the number of defibrillation shocks delivered, number of executed CPU cycles, number of switching power supply cycles used to charge an output capacitor for delivering shock pulses, a number reflecting the amount of data transmitted or received during RF telemetry sessions, number of ATP pacing pulses delivered, and/or the number of tachycardia episodes in which ATP therapy was delivered. Each event duration and event count may be related to a certain amount of charge consumption by an empirically determined charge coefficient, designated as A_{K} and B_{K}, respectively, for the duration or count of a particular event. The value of the charge coefficient for each particular type of event may be made to vary in accordance with changing or variable operating parameters of the device. For example, the charge coefficient used to calculate the charge consumption resulting from a bradycardia or ATP pace may be made to vary with pacing pulse amplitude, pulse width, and/or lead resistance. Similarly, the charge coefficient for calculating charge consumption produced by a shock pulse may be made to vary with shock pulse energy. If a charge coefficient for a particular type of event changes while the operational history of the device is being recorded, separate counts of events and event durations may be maintained for each value of the charge coefficient.

At step S3, the charge consumption CC over the time period T is estimated as:
CC=Σ A_{K}N_{K}+Σ B_{K}D_{K}
where the first summation is carried out from K=1 to n and the second summation from K=1 to m. At step S4, the calculated charge consumption CC over the time period T is added to the total charge consumption TCC calculated over previous time periods to give an updated total charge consumption:
TCC=TCC+CC
In certain embodiments, the total charge consumption TCC calculated as just described is used as is to estimate battery charge consumption. As an optional step, however, the total charge consumption TCC may be periodically adjusted based upon a coulombmeter measurement, a measured battery voltage and/or measured capacitor charge time in order to minimize error accumulation. This is shown at step S5 in the particular embodiment shown in FIG. 3, where the device calculates an estimated battery replacement time as a function of the estimated total charge consumption TCC. The device then initializes the event number and event duration variables at step S6 before returning to step S1 and recording the operational history for the next time period T.

The device may be additionally programmed to utilize the estimated charge consumption to alter its operation. For example, the device may be programmed to schedule automatic reforming of the output capacitor based upon the estimated charge consumption. The device could also be programmed to curtail certain activities or make only certain features available when the estimated charge consumption reaches a selected threshold value. For example, those features or activities of the device which are considered to be of a lower priority for a particular patient may be limited when the battery supply is low.

As noted above, in one embodiment, some of the processing described above may be performed by an external programmer or other device after transmission of data from the implantable device. The processing burden may be divided between the implantable device and external programmer in any arbitrarily selected manner. For example, the implantable device may transmit a total of the accumulated event durations and counts to an external programmer which then performs the calculations to estimate the total charge consumption and/or battery replacement time.

Although the invention has been described in conjunction with the foregoing specific embodiment, many alternatives, variations, and modifications will be apparent to those of ordinary skill in the art. Such alternatives, variations, and modifications are intended to fall within the scope of the following appended claims.

1. A cardiac rhythm management device, comprising:
one or more sensing channels for generating sense signals representing cardiac activity;
one or more pacing channels for delivering pacing therapy;
a battery for supplying power to the device;
a controller for interpreting sense signals and controlling the delivery of paces in accordance with a programmed pacing mode; and,
wherein the controller is further programmed to estimate the battery charge consumption by recording an operational history of the device which includes the number of occurrences or durations of selected events, multiplying each recorded event number or duration by a charge coefficient corresponding to the type of event, and summing the multiplication products.

2. The device of claim I further comprising circuitry for measuring the battery voltage and wherein the controller is further programmed to adjust the estimated charge consumption based upon the measured battery voltage.

3. The device of claim 1 further comprising:
an output capacitor charged by the battery for delivering defibrillation shocks;
circuitry for measuring the charge time of the output capacitor; and,
wherein the controller is further programmed to adjust the estimated charge consumption based upon the measured charge time.

4. The device of claim 1 further comprising a coulombmeter for measuring the battery charge consumption and wherein the controller is further programmed to adjust the estimated charge consumption based upon the coulombmeter measurement.

5. The device of claim 1 wherein the recorded operational history of the device includes the number of sensed events.

6. The device of claim 1 wherein the recorded operational history of the device includes the number or total duration of paces delivered in a bradycardia pacing mode.

7. The device of claim 1 further comprising means for delivering defibrillation shocks and wherein the recorded operational history of the device includes the number of shocks delivered or the cumulative charge time used to charge an output capacitor.

8. The device of claim 1 wherein the controller is further programmed to deliver anti-tachycardia pacing (ATP) therapy upon detection of an episode of tachycardia and wherein the recorded operational history of the device includes the number of tachycardia episodes in which ATP therapy was delivered.

9. The device of claim 1 further comprising a telemetry unit and wherein the recorded operational history of the device includes the measured time during which the telemetry unit was active.

10. The device of claim 1 wherein the recorded operational history of the device includes the elapsed time since the device was powered up.

11. The device of claim 1 wherein the recorded operational history of the device includes the duration of time in which the device was in a low-power storage mode.

12. The device of claim 1 further comprising a telemetry interface and wherein the controller is further programmed to transmit an indicator of battery status based upon the estimated charge consumption.

13. The device of claim 1 further comprising one or more hardware-based modalities for measuring a variable related to charge consumption and wherein the controller is programmed to compute an indicator of battery status which is the worst case of battery status as determined by the hardware-based modalities and the charge consumption estimated from operational history.

14. The device of claim 1 wherein a charge coefficient varies as particular operating parameters of the implantable device are changed.

15. The device of claim 1 wherein the controller is programmed to alter the operation of the device based upon the estimated charge consumption.

16. A system, comprising:
an implantable cardiac device which includes: one or more sensing channels for generating sense signals representing cardiac activity;
one or more pacing channels for delivering pacing therapy;
a battery for supplying power to the device;
a controller for interpreting sense signals and controlling the delivery of paces in accordance with a programmed pacing mode; and,
wherein the controller is further programmed to record an operational history of the device which includes the number of occurrences or durations of selected events;
an external programmer in communication with the implantable cardiac device via a telemetry link over which the recorded operational history of the device may be downloaded, wherein the external programmer is programmed to estimate the battery charge consumption by multiplying each recorded event number or duration by a charge coefficient corresponding to the type of event and summing the multiplication products.

17. The system of claim 16 further comprising circuitry for measuring the battery voltage and wherein the external programmer is further programmed to adjust the estimated charge consumption based upon the measured battery voltage.

18. The system of claim 16 further comprising:
an output capacitor charged by the battery for delivering defibrillation shocks;
circuitry for measuring the charge time of the output capacitor; and,
wherein the external programmer is further programmed to adjust the estimated charge consumption based upon the measured charge time.

19. The system of claim 16 further comprising a coulombmeter for measuring the battery charge consumption and wherein the external programmer is further programmed to adjust the estimated charge consumption based upon the coulombmeter measurement.

20. The system of claim 16 wherein the implantable device is equipped with one or more hardware-based modalities for measuring a variable related to charge consumption and wherein the external programmer is programmed to compute an indicator of battery status which is the worst case of battery status as determined by the hardware-based modalities and the charge consumption estimated from operational history.

21. A method for estimating battery charge consumption in a cardiac rhythm management device, comprising:
recording an operational history of the device which includes the number of occurrences or durations of selected events;
multiplying each recorded event number or duration by a charge coefficient corresponding to the type of event; and,
summing the multiplication products.

22. The method of claim 21 further comprising adjusting the estimated charge consumption based upon a hardware-based measurement related to battery charge consumption.

23. The method of claim 21 further comprising:
measuring a variable related to charge consumption with a hardware-based modality; and,
computing an indicator of battery status which is the worst case of battery status as determined by the hardware-based modality and the charge consumption estimated from operational history.

24. The method of claim 21 wherein the recorded operational history of the device includes the elapsed time since the device was powered up.

25. The method of claim 21 wherein the recorded operational history of the device includes the duration of time in which the device was in a low-power storage mode.
